(19) United States
(12) Patent Application Publication
(10) Pub. No.: US 2025/0325509 A1
(43) Pub. Date: Oct. 23, 2025

(54) POWDERS CONTAINING A BUFFER SALT AND AN AMINO ACID, RECONSTITUTION OF SUCH A POWDER INTO A NUTRITIONAL PRODUCT, AND METHODS OF USING SUCH A NUTRITIONAL PRODUCT

(71) Applicant: SOCIETE DES PRODUITS NESTLE S.A., Vevey (CH)

(72) Inventor: Seyhun Gemili, Bedminster, NJ (US)

(21) Appl. No.: 19/254,372

(22) Filed: Jun. 30, 2025

Related U.S. Application Data
(62) Division of application No. 17/290,517, filed on Apr. 30, 2021.
(60) Provisional application No. 62/754,918, filed on Nov. 2, 2018.

(51) Int. Cl. A61K 31/198; A23L 2/66; A23L 2/68; A23L 33/16; A23L 33/175; A61K 38/17; A61K 47/02

(52) U.S. Cl. CPC A61K 31/198; A23L 2/66; A23L 2/68; A23L 33/16; A23L 33/175; A61K 38/1709; A61K 47/02; A23V 2002/00

(57) ABSTRACT

A powder contains a buffer salt and an amino acid, for example one or both of (i) least one glycine or functional derivative thereof or (ii) at least one N-acetylcysteine, L-cysteine or functional derivative thereof. Optionally the powder additionally includes at least one vitamin or mineral. Preferably the powder has at least one characteristic selected from the group consisting of (i) the amino acid is present in an amount that at least partially comprises the amino acid in a form selected from the group consisting of free form, dipeptides, tripeptides and mixtures thereof and (ii) the powder is substantially free of enzymes and antibodies. The powder can be reconstituted in a liquid containing an acidic component, such as juice or a polyphenol, and/or a protein, such as milk protein, plant protein, collagen or blends thereof. The buffer salt can minimize or prevent denaturation of protein and/or coagulation of protein upon reconstitution of the powder in the liquid.

### CROSS REFERENCE TO RELATED APPLICATIONS

The present application is a divisional of patent application Ser. No. 17/290,517, filed on Apr. 30, 2021, which is a National Stage of International Application No. PCT/EP2019/079947, filed on Nov. 1, 2019, which claims priority to U.S. Provisional Patent Application No. 62/754,918, filed on Nov. 2, 2018, the entire contents of which are being incorporated herein by reference.

### DESCRIPTION

The present disclosure generally relates to compositions and methods that use buffer salt, amino acid, and optionally a vitamin and/or a mineral. In a non-limiting embodiment, the composition is a powder that comprises at least one glycine or functional derivative thereof, at least one N-acetyl-cysteine, L-cysteine or functional derivative thereof, and a buffer salt; and the powder can be reconstituted in a liquid to form a nutritional product.

Population aging has been a remarkable demographic event. As the growth of the older population has outpaced the total population due to increased longevity, the proportion of older persons relative to the rest of the population has increased considerably. For example, one in every twelve individuals was at least 60 years of age in 1950, and one in every ten was aged 60 years or older by the end of 2000. By the end of 2050, the number of persons worldwide that is 60 years or over is projected to be one in every five.

Aged or aging individuals frequently suffer some degree of cognitive impairment, including decline in cognitive function, that progresses with age, and age-related changes in brain morphology and cerebrovascular function are commonly observed. Cognitive decline has been consistently reported with aging across a range of cognitive domains including processing speed, attention, episodic memory, spatial ability and executive function. Brain imaging studies have revealed that these normal age-related cognitive declines are associated with decreases in both grey and white matter volume in the brain, with the fronto-striatal system most heavily compromised with aging. These decreases in cortical volume can be attributed to a number of detrimental cellular processes involved with normal aging, such as accumulation of damage by free radicals over time leading to oxidative damage, chronic low-grade inflammation, homocysteine accumulation (which when elevated are a risk factor for cognitive impairment and dementia), and decreased mitochondrial efficiency. In addition to direct cellular damage, the brain is also indirectly impaired by insults to micro-vascular structures.

It is evident that the pathology of aging and also dementia involves a complexity of these interacting factors which are linked together. For example, mitochondrial dysfunction leads to increased oxidative stress, and oxidative stress can trigger inflammation and vascular insults.

Furthermore, cognitive decline is an early predictor or Alzheimer pathology and begins before the onset of dementia. In this context, the cognitive composite score represents a reliable means to assess the cognitive decline preceding dementia. Considerable evidence suggests that maintaining brain health and preventing cognitive decline with advancing age may prevent or delay development of dementia due to Alzheimer's disease and other aged related neuropathologies.

In biology and psychology, the term “stress” refers to the consequence of the failure of a human or other animal to respond appropriately to physiological, emotional, or physical threats, whether actual or imagined. The psychobiological features of stress may present as manifestations of oxidative stress, i.e., an imbalance between the production and manifestation of reactive oxygen species and the ability of a biological system readily to detoxify the reactive intermediates or to repair the resulting damage. Disturbances in the normal redox state of tissues can cause toxic effects through the production of peroxides and free radicals that damage all of the components of the cell, including proteins, lipids, and DNA. Some reactive oxidative species can even act as messengers through a phenomenon called “redox signaling.”

In humans, oxidative stress is involved in many diseases. Examples include atherosclerosis, Parkinson's disease, heart failure, myocardial infarction, Alzheimer's disease, schizophrenia, bipolar disorder, fragile X syndrome, and chronic fatigue syndrome.

One source of reactive oxygen under normal conditions in humans is the leakage of activated oxygen from mitochondria during oxidative phosphorylation. Other enzymes capable of producing superoxide (O2−) are xanthine oxidase, NADPH oxidases and cytochromes P450. Hydrogen peroxide, another strong oxidizing agent, is produced by a wide variety of enzymes including several oxidases. Reactive oxygen species play important roles in cell signaling, a process termed redox signaling. Thus, to maintain proper cellular homeostasis a balance must be struck between reactive oxygen production and consumption.

Oxidative stress contributes to tissue injury following irradiation and hyperoxia. It is also suspected to be important in neurodegenerative diseases, including Alzheimer's disease, Parkinson's disease, amyotrophic lateral sclerosis (ALS), and Huntington's disease.

Oxidative stress is also thought to be linked to certain cardiovascular diseases, since oxidation of low-density lipoprotein (LDL) in the vascular endothelium is a precursor to plaque formation. Oxidative stress also plays a role in the ischemic cascade due to oxygen reperfusion injury following hypoxia. This cascade includes both strokes and heart attacks. Oxidative stress has also been implicated in chronic fatigue syndrome.

Moreover, the free radical theory of aging suggests that the biological process of aging results in increased oxidative stress in elderly humans. The ability of a cell to resist the damaging potential of oxidative stress is determined by a vital balance between generation of oxidant free radicals and the defensive array of antioxidants available to the cell. There are multiple antioxidant defense systems and of these, glutathione (GSH) is the most abundant intracellular component of overall antioxidant defenses. GSH, a tripeptide, is synthesized from precursor amino-acids glutamate, cysteine, and glycine in two steps catalyzed by glutamate cysteine ligase (GCL, also known as gamma-glutamylcysteine synthetase, EC 6.3.2.2) and gamma-L-glutamyl-L-cysteine:glycine ligase (also known as glutathione synthetase, EC 6.3.2.3), and GSH synthesis occurs de novo in cells.

### SUMMARY

Acidic amino acids such as N-acetyl cysteine (NAC) can denature proteins in liquid. Existing solutions use basic additives such as potassium hydroxide and sodium hydroxide to increase the pH of protein-containing liquids. However, the present inventors noted that the increase in the pH of the protein-containing solution can change the configuration of the protein structures, which can lead to denaturation or gelation before the acidic amino acids are added.

The present inventors surprisingly found that buffering salts such as disodium phosphate and/or potassium phosphate dibasic can be blended with acidic amino acids and then added into liquids containing protein such as milk protein, plant protein, collagen or blends thereof. Thus denaturation of the proteins in different concentrations and conditions can be avoided by establishing and/or maintaining a stable pH of the liquid.

Indeed, proteins are sensitive to pH changes in the liquid environment, and even slight changes in the pH may cause disruption of protein structure and cause denaturation that could lead to gelation, precipitation and aggregation. Addition of acidic or basic components is one way that causes change in the pH of the environment. For example, amino acids have a chemical structure that could create the increase or decrease in the pH. Therefore, addition of these amino acids into protein containing liquid products could lead to denaturation due to increase or decrease of the pH. As set forth later herein, the present inventors evaluated addition of buffering salts to avoid drastic changes in the pH of protein containing liquid. The present inventors found that these buffering salts minimized the change in the pH and decreased the protein precipitation compared to cases at which no buffering salts were used.

Accordingly, in a general embodiment, the present disclosure provides a powder comprising a buffer salt and amino acid. The powder has at least one characteristic selected from the group consisting of (i) the amino acid is present in an amount that at least partially comprises the amino acid in a form selected from the group consisting of free form, dipeptides, tripeptides and mixtures thereof and (ii) the powder is substantially free of enzymes and antibodies.

In an embodiment, the amino acid comprises an acidic amino acid.

In an embodiment, the powder further comprises at least one vitamin or mineral.

In an embodiment, the powder further comprises at least one of calcium, sodium or potassium.

In an embodiment, the amino acid comprises L-cysteine, glycine, and L-glutamate that are provided by glutathione in the powder.

In an embodiment, the amino acid comprises at least one N-acetyl-cysteine or functional derivative thereof.

In an embodiment, the amino acid comprises a combination of at least one N-acetyl-cysteine or functional derivative thereof and at least one glycine or functional derivative thereof.

The powder can be in a unit dosage form comprising the combination of the at least one N-acetyl-cysteine, L-cysteine or functional derivative thereof and the at least one glycine or functional derivative thereof in an amount effective for treating, reducing incidence of, or reducing severity of at least one condition selected from the group consisting of metabolic syndrome, age-related decline in metabolic regulation, and muscle indications.

The powder can be in a unit dosage form comprising the combination of the at least one N-acetyl-cysteine, L-cysteine or functional derivative thereof and the at least one glycine or functional derivative thereof in an amount effective for at least one of (i) treating or preventing at least one physical state selected from the group consisting of oxidative stress, a condition associated with oxidative stress (e.g., aging and its effects such as skin aging), a reduced level of glutathione, a condition associated with a reduced level of glutathione, or (ii) improving one or more of fetal metabolic programming for prevention of later development of obesity, pre-diabetes and/or diabetes, maternal and fetal health in gestational diabetes, exercise capacity and physical function, quality of life, longevity, memory, cognition, post-traumatic recovery and survival, or recovery from trauma and surgery.

The powder can be in a unit dosage form comprising the combination of the at least one N-acetyl-cysteine or functional derivative thereof and the at least one glycine or functional derivative thereof in an amount effective to treat or prevent at least one physical state selected from the group consisting of deleterious effects of aging, muscle loss, pre-diabetes, gestational diabetes, type I diabetes, type II diabetes, complications from diabetes, insulin resistance, metabolic syndrome, dyslipidemia, overweight, obesity, raised cholesterol levels, raised triglyceride levels, elevated fatty acid levels, fatty liver disease, renal disease, cardiovascular disease, neurodegenerative disease, impaired cognitive function, myopathy such as statin-induced myopathy, non-alcoholic steatohepatitis, tinnitus, dizziness, alcohol hangover, hearing impairment, osteoporosis, hypertension, atherosclerosis/coronary artery disease, myocardial damage after stress, traumatic brain injury, cystic fibrosis, inflammation, cancer, and HIV infection.

In an embodiment, the powder is substantially free of proteins having at least four amino acid residues.

In another embodiment, the present disclosure provides a method of decreasing or preventing protein denaturation and/or protein coagulation cause by reconstitution of a powder in a liquid comprising an ingredient selected from the group consisting of a protein, an acidic component, and a mixture thereof. The method comprises a step selected from the group consisting of (i) dry mixing a buffer salt with an amino acid and (ii) drying an aqueous solution comprising a buffer salt and an amino acid. The powder has at least one characteristic selected from the group consisting of (i) the amino acid is present in an amount that at least partially comprises the amino acid in a form selected from the group consisting of free form, dipeptides, tripeptides and mixtures thereof and (ii) the powder is substantially free of enzymes and antibodies.

In an embodiment, the amino acid comprises at least one N-acetyl-cysteine or functional derivative thereof.

In an embodiment, the amino acid comprises a combination of at least one N-acetyl-cysteine, L-cysteine or functional derivative thereof and at least one glycine or functional derivative thereof.

In an embodiment, the powder further comprises at least one vitamin or mineral.

In another embodiment, the present disclosure provides a liquid nutritional product comprising a buffer salt, an amino acid, and an ingredient selected from the group consisting of an acidic component, a protein and mixtures thereof. The nutritional product is made by a process comprising reconstituting a powder comprising the buffer salt and the amino acid into a liquid comprising the ingredient. The powder has at least one characteristic selected from the group consisting of (i) the amino acid is present in an amount that at least partially comprises the amino acid in a form selected from the group consisting of free form, dipeptides, tripeptides and mixtures thereof and (ii) the powder is substantially free of enzymes and antibodies.

In an embodiment, the nutritional product is a beverage.

In an embodiment, the liquid comprises an acidic component that is at least one of juice or a polyphenol (e.g., a flavonoid).

In an embodiment, the liquid comprises a protein selected from the group consisting of milk protein, plant protein, collagen and blends thereof.

In another embodiment, the present disclosure provides a method of making a powder formulated for reconstitution into a liquid to form a nutritional product. The liquid comprises an ingredient selected from the group consisting of a protein, an acidic component, and a mixture thereof. The method comprises a step selected from the group consisting of (i) dry mixing a buffer salt with an amino acid and (ii) drying an aqueous solution comprising a buffer salt and an amino acid. The powder has at least one characteristic selected from the group consisting of (i) the amino acid is present in an amount that at least partially comprises the amino acid in a form selected from the group consisting of free form, dipeptides, tripeptides and mixtures thereof and (ii) the powder is substantially free of enzymes and antibodies.

In an embodiment, the amino acid comprises at least one N-acetyl-cysteine, L-cysteine or functional derivative thereof.

In an embodiment, the amino acid comprises a combination of at least one N-acetyl-cysteine, L-cysteine or functional derivative thereof and at least one glycine or functional derivative thereof.

In an embodiment, the powder further comprises at least one vitamin or mineral.

An advantage of one or more embodiments provided by the present disclosure is to facilitate reconstitution of an amino acid, such as N-acetyl cysteine, L-cysteine and/or glycine, by minimizing or preventing protein denaturation and coagulation in a liquid in which the amino acid is reconstituted, such as a liquid containing an acidic component that is at least one of juice or a polyphenol (e.g., a flavonoid) and/or containing protein such as milk protein, plant protein, collagen or blends thereof.

Another advantage of one or more embodiments provided by the present disclosure is to facilitate reconstitution of a powder containing a vitamin and/or a mineral that can increase precipitation, such as sodium, potassium or calcium, by minimizing or preventing the negative impact of such compounds on the dissolution of the powder in a liquid.

Yet another advantage of one or more embodiments provided by the present disclosure is to replenish Glutathione pools, which decline with age.

An advantage of one or more embodiments provided by the present disclosure is to help off-set slowing of the metabolism associated with aging.

Another advantage of one or more embodiments provided by the present disclosure is to help increase fatty acids metabolism.

An advantage of one or more embodiments provided by the present disclosure is to help support healthy LDL-cholesterol and fatty acid levels in the blood.

Another advantage of one or more embodiments provided by the present disclosure is to supplement key amino acids which become less available in cells in sufficient quantities during aging.

Yet another advantage of one or more embodiments provided by the present disclosure is to provide amino acids that are precursors to the production of Glutathione, which is important for cellular function and cellular protection.

An advantage of one or more embodiments provided by the present disclosure is to help increase Glutathione levels within cells.

Another advantage of one or more embodiments provided by the present disclosure is to improve concentration of Glutathione levels which decline with age.

Yet another advantage of one or more embodiments provided by the present disclosure is to help maintain healthy muscle mass.

An advantage of one or more embodiments provided by the present disclosure is to help reduce oxidative stress on the body.

Another advantage of one or more embodiments provided by the present disclosure is to support a normal immune system via Glutathione modulation.

Additional features and advantages are described herein and will be apparent from the following Figures and Detailed Description.

### BRIEF DESCRIPTION OF DRAWINGS

FIG. 1 shows the experimentally tested dissolution of powdered N-acetyl-cysteine (NAC) without buffer salts in a dairy-based liquid at room temperature as disclosed herein.

FIG. 2 shows the experimentally tested dissolution of powdered NAC with buffer salts in a dairy-based liquid at room temperature as disclosed herein.

FIG. 3 shows the experimentally tested dissolution of powdered NAC and glycine without buffer salts in a dairy-based liquid at room temperature as disclosed herein.

FIG. 4 shows the experimentally tested dissolution of powdered NAC and glycine with buffer salts in a dairy-based liquid at room temperature as disclosed herein.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified. A composition “consisting essentially of” contains at least 50 wt. % of the referenced components, preferably at least 75 wt. % of the referenced components, more preferably at least 85 wt. % of the referenced components, most preferably at least 95 wt. % of the referenced components.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “at least one glycine or functional derivative thereof” should be interpreted as “glycine,” or “a functional derivative of glycine,” or “both glycine and a functional derivative of glycine.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The term “nutritional product” as used herein means a composition that is intended for ingestion by an individual such as a human and provides at least one nutrient to the individual. The nutritional products of the present disclosure, including the many embodiments described herein, can comprise, consist of, or consist essentially of the elements disclosed herein, as well as any additional or optional ingredients, components, or elements described herein or otherwise useful in a diet.

As used herein, the term “isolated” means removed from one or more other compounds or components with which the compound may otherwise be found, for example as found in nature. For example, “isolated” preferably means that the identified compound is separated from at least a portion of the cellular material with which it is typically found in nature. In an embodiment, an isolated compound is pure, i.e., free from any other compound.

“Prevention” includes reduction of risk and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

The term “unit dosage form,” as used herein, refers to physically discrete units suitable as unitary dosages for human and animal subjects, each unit containing a predetermined quantity of the composition disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of powder in a sachet.

A “subject” or “individual” is a mammal, preferably a human. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly individuals.

As used herein, an “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects of the composition disclosed herein, namely a composition comprising at least one glycine or functional derivative thereof, at least one N-acetylcysteine, L-cysteine or functional derivative thereof, and buffer salt, relative to a composition lacking one or more of these compounds but otherwise identical. As used herein, “promoting” refers to enhancing or inducing a particular level relative to the level before administration of the composition disclosed herein.

“Sarcopenia” is defined as the age-associated loss of muscle mass and functionality (including muscle strength and gait speed). As used herein, “frailty” is defined as a clinically recognizable state of increased vulnerability resulting from aging-associated decline in reserve and function across multiple physiologic systems such that the ability to cope with everyday or acute stressors is compromised. In the absence of an established quantitative standard, frailty has been operationally defined by Fried et al. as meeting three out of five phenotypic criteria indicating compromised energetics: (1) weakness (grip strength in the lowest 20% of population at baseline, adjusted for gender and body mass index), (2) poor endurance and energy (self-reported exhaustion associated with VO₂ max), (3) slowness (lowest 20% of population at baseline, based on time to walk 15 feet, adjusting for gender and standing height), (4) low physical activity (weighted score of kilocalories expended per week at baseline, lowest quintile of physical activity identified for each gender; e.g., less than 383 kcal/week for males and less than 270 kcal/week for females), and/or unintentional weight loss (10 lbs. in past year). Fried L P, Tangen C M, Walston J, et al., “Frailty in older adults: evidence for a phenotype.” J. Gerontol. A. Biol. Sci. Med. Sci. 56(3):M146-M156 (2001). A pre-frail stage, in which one or two of these criteria are present, identifies a high risk of progressing to frailty.

“Cachexia” is a severe body wasting condition characterized by marked weight loss, anorexia, asthenia, and anaemia. Cachexia is a common feature of a number of illnesses, such as cancer, sepsis, chronic heart failure, rheumatoid arthritis, and acquired immune deficiency syndrome (AIDS).

“Overweight” is defined for a human as a body mass index (BMI) between 25 and 30 kg/m². “Obese” is defined for a human as a BMI of at least 30 kg/m², for example 30-39.9 kg/m². “Weight loss” is a reduction of the total body weight. Weight loss may, for example, refer to the loss of total body mass in an effort to improve one or more of health, fitness or appearance.

“Diabetes” encompasses both the type I and type II forms of the disease. Non-limiting examples of risk factors for diabetes include: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

As used herein, the term “metabolic syndrome” refers to a combination of medical disorders that, when occurring together, increase the risk of developing cardiovascular disease and diabetes. It affects one in five people in the United States and prevalence increases with age. Some studies have shown the prevalence in the United States to be an estimated 25% of the population. In accordance with the International Diabetes Foundation consensus worldwide definition (2006), metabolic syndrome is central obesity plus any two of the following:
Raised triglycerides: >150 mg/dL (1.7 mmol/L), or specific treatment for this lipid abnormality;
Reduced HDL cholesterol: <40 mg/dL (1.03 mmol/L) in males, <50 mg/dL (1.29 mmol/L) in females, or specific treatment for this lipid abnormality;
Raised blood pressure: systolic BP>130 or diastolic BP>85 mm Hg, or treatment of previously diagnosed hypertension; and
Raised fasting plasma glucose: (FPG)>100 mg/dL (5.6 mmol/L), or previously diagnosed type 2 diabetes.

As used herein, “neurodegenerative disease” or “neurodegenerative disorder” refers to any condition involving progressive loss of functional neurons in the central nervous system. In an embodiment, the neurodegenerative disease is associated with age-related cell death. Non-limiting examples of neurodegenerative diseases include Alzheimer's disease, Parkinson's disease, Huntington's disease, amyotrophic lateral sclerosis (also known as ALS and as Lou Gehrig's disease), AIDS dementia complex, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome.

As used herein, “cognitive performance” refers to how well a subject performs one or more cognitive function. As used herein, “cognitive function” refers to any mental process that by which one becomes aware of, perceives, or comprehends ideas. It involves symbolic operations, e.g., all aspects of perception, thinking, reasoning, and remembering and includes, for example, perception, memory, attention, speech comprehension, speech generation, reading comprehension, creation of imagery, learning, and reasoning, preferably. Ordinarily it will refer to at least memory.

Methods for measuring cognitive function are well-known and can include, for example, individual or battery tests for any aspect of cognitive function. One such test is the Prudhoe Cognitive Function Test by Margallo-Lana et al. (2003) J. Intellect. Disability Res. 47:488-492. Another such test is the Mini Mental State Exam (MMSE), which is designed to assess orientation to time and place, registration, attention and calculation, recall, language use and comprehension, repetition, and complex commands. Folstein et al. (1975) J. Psych. Res. 12:189-198. Other tests useful for measuring cognitive function include the Alzheimer Disease Assessment Scale-Cognitive (ADAS-Cog) (Rosen et al. (1984) Am. J. Psychiatry. 141(11):1356-64) and the Cambridge Neuropsychological Test Automated Battery (CANTAB) (Robbins et al. (1994) Dementia. 5(5):266-81). Such tests can be used to assess cognitive function in an objective manner, so that changes in cognitive function, for example in response to treatment in accordance with methods disclosed herein, can be measured and compared.

As used herein, a “cognitive disorder” refers to any condition that impairs cognitive function. Non-limiting examples of a cognitive disorder include delirium, dementia, learning disorder, attention deficit disorder (ADD), and attention deficit hyperactivity disorder (ADHD).

As used herein, “amino acid” includes the standard (proteinogenic) amino acids and also derivatives of the standard amino acids, such as compounds resulting from reaction at an amino group, carboxy group, or side-chain functional group of the amino acid or from the replacement of any hydrogen by a heteroatom. Non-limiting examples of suitable amino acid derivatives according to the present disclosure include amino acid esters such as methyl esters, amino acid tert-butyl esters, benzyl esters, and ethyl esters. An “acidic” amino acid is any amino acid or derivative thereof that is capable of donating a hydron (proton or hydrogen ion H+) or forming a covalent bond with an electron pair.

As used herein, a “powder” is a composition in which any moisture is no greater than 6 wt. % of the composition.

### Embodiments

An aspect of the present disclosure is a powder formulated for reconstitution in a liquid to form a nutritional product, the powder comprising a buffer salt, an amino acid, and optionally at least one vitamin or mineral. In an embodiment, the amino acid comprises one or both of (i) at least one glycine or functional derivative thereof or (ii) at least one N-acetylcysteine, L-cysteine or functional derivative thereof. In an embodiment, the amino acid comprises L-cysteine, L-glutamic acid, and glycine that are provided by glutathione in the powder.

Non-limiting examples of suitable vitamins include vitamin C and group B vitamins, and other non-limiting examples of suitable vitamins include ascorbic acid, ascorbyl palmitate, vitamins B1, B2, B6, B12, and Niacin (B3), or combination of thereof. The vitamins may also include Vitamins A, D, E and K and acid vitamins such as pantothenic acid, folic acid and biotin. The Vitamin A may be present as Vitamin A Palmitate and/or beta-carotene. Vitamin D3 is an example of a suitable form of Vitamin D, and Vitamin D2 can also be used in some embodiments.

Non-limiting examples of suitable minerals include calcium, sodium, magnesium, iron, zinc, iodine, copper, phosphorus, manganese, potassium, chromium, molybdenum, selenium, nickel, tin, silicon, vanadium and boron.

The powder can be administered in an amount that provides the recommended daily requirement (RDA) of the vitamin and/or mineral therein. In some embodiments, a unit dosage form of the powder contains a predetermined portion of the RDA for the vitamin and/or mineral, for example about 50% of the RDA, about 75% of the RDA, about 100% of the RDA, or values therebetween. For example, a unit dosage form of the powder can contain about 50% of the RDA for the vitamin and/or mineral such that administration of two of the unit dosage forms in one day provides approximately the entirety of the RDA for the corresponding vitamin and/or mineral. As another example, a unit dosage form of the powder can contain 100% of the RDA for the vitamin and/or mineral such that administration of one of the unit dosage forms provides approximately the entirety of the RDA for the corresponding vitamin and/or mineral.

A preferred mixture of vitamins and minerals comprises Vitamin E, Vitamin C, Vitamin B2 and Zinc. Therefore, a preferred embodiment of the powder comprises a buffer salt, at least one glycine or functional derivative thereof, at least one N-acetylcysteine or functional derivative thereof, Vitamin E, Vitamin C, Vitamin B2 and Zinc, most preferably with the vitamins and minerals administered in an amount within the RDA and/or present in the powder in an amount within the RDA.

A particularly preferred embodiment of the powder comprises a buffer salt comprising at least one phosphate salt, the powder further comprising at least one glycine or functional derivative thereof, at least one N-acetylcysteine or functional derivative thereof, Vitamin E, Vitamin C, Vitamin B2 and Zinc and/or Selenium, most preferably with the vitamins and minerals administered in an amount within the RDA and/or present in the powder in an amount within the RDA.

As a non-limiting example, a unit dosage form of the powder can comprise about 600 mg N-acetyl cysteine (NAC), about 600 mg Glycine, one or more phosphate salts (e.g., about 500 mg total phosphate salt), and the RDA of each of Vitamin E, Vitamin C, Vitamin B2 and Zinc.

As another non-limiting example, a unit dosage form of the powder can comprise about 485 mg N-acetyl cysteine (NAC), about 485 mg Glycine, one or more phosphate salts (e.g., about 500 mg total phosphate salt), and the RDA of each of Vitamin E, Vitamin C, Vitamin B2, Selenium and Zinc

Non-limiting examples of suitable buffer salts include citrates; phosphates such as monophosphates, diphosphates, triphosphates, and polyphosphates (e.g., magnesium phosphate and trisodium phosphate); hexametaphosphate; carbonates such as monocarbonates and bicarbonates (e.g., magnesium carbonate, sodium monocarbonate, sodium bicarbonate, potassium bicarbonate and potassium monocarbonate); and mixtures thereof. Preferably the buffer salt comprises at least one of dibasic sodium phosphate, monobasic potassium phosphate, dipotassium phosphate, monobasic calcium phosphate, dicalcium phosphate, or tricalcium phosphate; most preferably at least one of disodium phosphate or potassium phosphate dibasic. The present disclosure is not limited to a specific embodiment of the buffer salt, and the buffer salt can be any compound that can minimize or prevent a pH change upon addition of an acid or an alkali to an aqueous medium in which the compound is dispersed.

The powder can be formed by drying a liquid comprising the at least one glycine or functional derivative thereof, the at least one N-acetylcysteine or functional derivative thereof, the buffer salt and optionally at least one vitamin or mineral. The drying can comprise spray-drying, freeze-drying or any other procedure of drying known in the art. The present disclosure is not limited to a specific embodiment of the drying by which the powder is made, and the drying can be any process that decreases moisture, e.g., to form a solid composition. Additionally or alternatively, the powder can be made by dry mixing.

In a preferred embodiment, the powder has at least one characteristic selected from the group consisting of (i) the amino acid is present in an amount that at least partially comprises the amino acid in a form selected from the group consisting of free form, dipeptides, tripeptides and mixtures thereof and (ii) the powder is substantially free of enzymes and antibodies. For example, at least a portion of the amino acids can be free form, i.e., not covalently bound to any other compound. Additionally or alternatively, at least a portion of the amino acids can be present in one or more of di-peptides; tri-peptides; larger peptides (i.e., at least four amino acid residues therein) or intact proteins. Nevertheless, in a preferred embodiment, the powder is substantially free of therapeutic proteins such as enzymes and/or antibodies, and in a particular embodiment, completely free of therapeutic proteins such as enzymes and/or antibodies. In some embodiments, the powder is substantially free (or completely free) of proteins of four or more amino acid residues. “Substantially free” means less than 1.0 wt. %, preferably less than 0.5 wt. %, more preferably less than 0.2 wt. %, even more preferably less than 0.1 wt. %, most preferably less than 0.01 wt. %. In some embodiments, the powder consists essentially of (or consists of) any residual water (e.g., after drying), the buffer salt, the amino acid, and optionally at least one vitamin or mineral.

The powder can be reconstituted in a liquid such as water to form a nutritional product such as a beverage, preferably at room temperature and/or without applying heat. In a preferred embodiment, the powder can be reconstituted in a liquid comprising a component selected from the group consisting of an acidic component (e.g., a juice and/or a polyphenol such as a flavonoid) and a protein (e.g., milk protein such as casein and/or whey; plant protein; collagen; and blends thereof). For example, the powder can be reconstituted in an oral nutrition supplement (ONS) such as the commercially available products MERITENE®, BOOST®, NUTREN® and SUSTAGEN®. Nevertheless, the present disclosure is not limited to a specific embodiment of the liquid in which the powder is reconstituted, and the liquid can be any liquid suitable for consumption by an animal or human.

Further in this regard, the present disclosure is not limited to a specific embodiment of the acidic component in the liquid, and the acidic component can be any compound that provides acidity to the liquid. Still further, the present disclosure is not limited to a specific embodiment of the protein in the liquid, and the protein can be any protein, particularly those types that are subject to denaturation or gelation upon a pH increase.

The powder can be provided to the consumer in a container (e.g., a sealed container) for reconstitution in the container and/or for allowing the user to pour the powder from the container into a drinking receptacle in which the powder is reconstituted. Non-limiting examples of suitable containers include bags, boxes, cartons, bottles, or combinations thereof. Preferred containers include a sachet/stick pack, i.e., a small disposable pouch, typically of flexible film such as cellophane or paper, preferably capable of being torn open at one or both ends, and containing one unit dosage form of the powder (i.e., one serving of the powder).

The combination of at least one glycine or functional derivative thereof and at least one N-acetylcysteine or functional derivative thereof can be provided by any of the compositions disclosed by U.S. Pat. Nos. 8,362,080, 8,802,730 and 9,084,760, each entitled “Increasing glutathione levels for therapy,” and U.S. Patent App. Pub. No. 2018/0161297 entitled “Benefits of Supplementation with N-Acetylcysteine and Glycine to Improve Glutathione Levels,” each incorporated herein by reference in its entirety.

In an embodiment, a unit dosage form of the powder comprises the combination of at least one glycine or functional derivative thereof and at least one N-acetylcysteine, L-cysteine or functional derivative thereof in an amount effective for treatment or prevention of at least condition selected from the group consisting of deleterious effects of aging, muscle loss (for any reason, including at least sarcopenia, HIV infection, aging, cachexia, deleterious effects of weightlessness), pre-diabetes, gestational diabetes, diabetes (type I or type II), complications from diabetes (e.g., diabetic dyslipidemia and/or diabetic microvascular complications such as nephropathy, retinopathy, and/or neuropathy), insulin resistance, metabolic syndrome, dyslipidemia, overweight, obesity, raised cholesterol levels, raised triglyceride levels, elevated fatty acid levels, fatty liver disease (e.g., non-alcoholic fatty liver disease, including with or without inflammation), renal disease, cardiovascular disease (e.g., heart failure and/or impaired cardiac contractile function, for example by treating heat failure and/or reducing severity or incidence of heart failure), neurodegenerative disease (e.g., from aging), impaired cognitive function, myopathy such as statin-induced myopathy, non-alcoholic steatohepatitis, tinnitus, dizziness, alcohol hangover, hearing impairment, osteoporosis, hypertension, atherosclerosis/coronary artery disease, myocardial damage after stress (e.g., from burns or trauma), traumatic brain injury (including concussions), cystic fibrosis, inflammation, cancer, and HIV infection. Further regarding aging, it is notable that reduction of glutathione can accompany aging. Further regarding cardiovascular disease (CVD), it is notable that increased homocysteine is a validated risk factor for CVD.

Another aspect of the present disclosure is a liquid nutritional product comprising a buffer salt, an amino acid, and an ingredient selected from the group consisting of an acidic component, a protein and mixtures thereof, the nutritional product made by a process comprising reconstituting a powder comprising the buffer salt and the amino acid into a liquid comprising the ingredient. Preferably the liquid nutritional product is a beverage. The reconstitution can be performed at room temperature and/or without applying heat.

Another aspect of the present disclosure is a method of treating at least one of these conditions, the method comprising administering to the individual a therapeutically effective amount of a combination of at least one glycine or functional derivative thereof and at least one N-acetylcysteine or functional derivative thereof in a nutritional product reconstituted from a powder comprising the combination and further comprising a buffer salt. Another aspect of the present disclosure is a method of preventing at least one of these conditions, the method comprising administering to an individual at risk of the at least one condition a prophylactically effective amount of a combination of at least one glycine or functional derivative thereof and at least one N-acetylcysteine, L-cysteine or functional derivative thereof in a nutritional product reconstituted from a powder comprising the combination and further comprising a buffer salt.

The powder and/or the resultant nutritional product can treat or prevent sarcopenia, sarcopenic obesity, or cachexia, for example cachexia from an underlying medical condition such as chronic illness, HIV, cancer, chronic obstructive pulmonary disease (COPD), and/or aging in otherwise healthy individuals. In this regard, aging can be accompanied by reduction of glutathione (GSH).

The powder and/or the resultant nutritional product can treat or prevent an eye condition resulting directly or indirectly from low GSH levels, including low levels in the lens of the eye that is known for being rich in glutathione. Non-limiting examples of such conditions include cataracts and/or glaucoma, presbyopia (loss of near vision with aging requiring reading glasses), and presbyacusis (loss of hearing with aging, which requires a hearing aid).

In an embodiment, the powder and/or the resultant nutritional product improves at least one of muscle performance or muscle recovery, such as from muscle stress, including muscle stress associated with exercise. The exercise may be of any kind, including aerobic (“cardio”) exercise and/or weight training, for example. The nutritional product can be administered during at least one time selected from the group consisting of before the exercise (e.g., less than one hour before), during the exercise, and after the exercise (e.g., less than one hour after the exercise).

Yet another aspect of the present disclosure is a method of delaying off-set of metabolic decline, maintaining muscle mass, decreasing oxidative stress, maintaining immune function and/or maintaining cognitive function in a healthy older adult. The method comprises administering to the healthy older adult an effective amount of a combination of at least one glycine or functional derivative thereof and at least one N-acetylcysteine, L-cysteine or functional derivative thereof in a nutritional product reconstituted from a powder comprising the combination and further comprising a buffer salt.

Another aspect of the present disclosure is a method of improving mitochondrial function in an individual with sarcopenia. The method comprises administering to the individual an effective amount of a combination of at least one glycine or functional derivative thereof and at least one N-acetylcysteine, L-cysteine or functional derivative thereof in a nutritional product reconstituted from a powder comprising the combination and further comprising a buffer salt.

Yet another aspect of the present disclosure is a method of enhancing metabolizing of reactive oxygen species, improving glucose control and/or improving muscle function in an individual with at least one of obesity, pre-diabetes or diabetes. The method comprises administering to the individual an effective amount of a combination of at least one glycine or functional derivative thereof and at least one N-acetylcysteine, L-cysteine or functional derivative thereof in a nutritional product reconstituted from a powder comprising the combination and further comprising a buffer salt.

Another aspect of the present disclosure is a method of improving mitochondrial function (preferably to benefit at least one of metabolism or strength) in an individual with sarcopenia. The method comprises administering to the individual an effective amount of a combination of at least one glycine or functional derivative thereof and at least one N-acetylcysteine, L-cysteine or functional derivative thereof in a nutritional product reconstituted from a powder comprising the combination and further comprising a buffer salt.

In another aspect, the present disclosure provides a method of improving cognitive function. The method comprises administering to an individual an effective amount of aa combination of at least one glycine or functional derivative thereof and at least one N-acetylcysteine, L-cysteine or functional derivative thereof in a nutritional product reconstituted from a powder comprising the combination and further comprising a buffer salt. The cognitive function can be selected from the group consisting of perception, memory, attention, speech comprehension, speech generation, reading comprehension, creation of imagery, learning, reasoning, and combinations thereof. In an embodiment, the individual does not have a cognitive disorder; alternatively, the individual has a cognitive disorder. The individual can be elderly and/or can have cognitive decline associated with aging.

Yet another aspect of the present disclosure is a method of improving one or more of fetal metabolic programming for prevention of later development of obesity, pre-diabetes and/or diabetes, maternal and fetal health in gestational diabetes, exercise capacity and physical function, quality of life, longevity, memory, cognition, post-traumatic recovery and survival (e.g., post-surgical, post-sepsis, post-blunt or penetrating trauma due to accident or physical assault), or recovery from trauma and surgery. The method comprises administering to the individual an effective amount of a combination of at least one glycine or functional derivative thereof and at least one N-acetylcysteine or functional derivative thereof in a nutritional product reconstituted from a powder comprising the combination and further comprising a buffer salt.

In another aspect, the present disclosure provides a method of achieving at least one result selected from the group consisting of (i) reducing severity and/or incidence of effects of aging, (ii) maintaining or improving cellular functioning and/or overall health, (iii) supporting at least one of normal mitochondrial function, cellular protection, or energy metabolism, (iv) increasing daily energy level, (v) reducing fatigue, (vi) maintaining or improving physical energy and/or cognitive performance, (vii) promoting healthy aging by promoting healthy or normal cellular function, (viii) supporting healthy skin, (ix) treating heat failure and/or reducing severity or incidence of heart failure, (x) treating, reducing incidence of, or reducing severity of oxidative stress and/or reduced glutathione (GSH) experienced during a time period comprising a stay in an intensive care unit (ICU), (xi) treating, reducing incidence of, or reducing severity of another condition associated with oxidative stress and/or reduced GSH (e.g., aging and its effects such as skin aging), (xii) promoting rehabilitation from injury, illness or surgery, for example rehabilitation specific to neurological conditions such as traumatic brain injury or stroke, (xiii) modulating glutathione levels in a patient having cancer or in remission from cancer, (xiv) treating, reducing incidence of, or reducing severity of symptoms from bariatric surgery, (xv) treating, reducing incidence of, or reducing severity of non-alcoholic fatty liver disease (NAFLD), (xvi) treating, reducing incidence of, or reducing severity of human immunodeficiency virus infection (HIV), and (xvii) combinations thereof, the method comprising administering to an individual an effective amount of a combination of at least one glycine or functional derivative thereof and at least one N-acetylcysteine or functional derivative thereof in a nutritional product reconstituted from a powder comprising the combination and further comprising a buffer salt.

A particularly preferred embodiment is a method of treating, reducing incidence of, or reducing severity of at least one condition selected from the group consisting of metabolic syndrome, age-related decline in metabolic regulation, and muscle indications, the method comprising administering to an individual in need thereof or at risk thereof an effective amount of a combination of at least one glycine or functional derivative thereof and at least one N-acetylcysteine or functional derivative thereof in a nutritional product reconstituted from a powder comprising the combination and further comprising a buffer salt.

Another aspect of the present disclosure is a method of treating, reducing an incidence of, reducing a severity of, and/or preventing overweight and/or obesity, the method comprising: orally administering to an individual an effective amount of a nutritional product comprising at least one glycine or functional derivative thereof, at least one N-acetylcysteine or functional derivative thereof, a buffer salt, and an ingredient selected from the group consisting of a protein, an acidic component, and a mixture thereof, wherein the nutritional product has been produced before the administering by a process comprising reconstituting a powder into a liquid comprising the ingredient, the powder comprising the at least one glycine or functional derivative thereof, the at least one N-acetylcysteine. L-cysteine or functional derivative thereof, and the buffer salt.

Another aspect of the present disclosure is a method of treating, reducing an incidence of, reducing a severity of, and/or preventing skin aging, the method comprising: orally administering to an older adult or elderly individual an effective amount of a nutritional product comprising at least one glycine or functional derivative thereof, at least one N-acetylcysteine or functional derivative thereof, a buffer salt, and an ingredient selected from the group consisting of a protein, an acidic component, and a mixture thereof, wherein the nutritional product has been produced before the administering by a process comprising reconstituting a powder into a liquid comprising the ingredient, the powder comprising the at least one glycine or functional derivative thereof, the at least one N-acetylcysteine or functional derivative thereof, and the buffer salt.

Another aspect of the present disclosure is a method of promoting healthy skin and/or treating, reducing an incidence of, reducing a severity of, and/or preventing a skin condition or a skin disease, the method comprising: orally administering to a human less than 45 years of age an effective amount of a nutritional product comprising at least one glycine or functional derivative thereof, at least one N-acetylcysteine or functional derivative thereof, a buffer salt, and an ingredient selected from the group consisting of a protein, an acidic component, and a mixture thereof, wherein the nutritional product has been produced before the administering by a process comprising reconstituting a powder into a liquid comprising the ingredient, the powder comprising the at least one glycine or functional derivative thereof, the at least one N-acetylcysteine or functional derivative thereof, and the buffer salt.

Another aspect of the present disclosure is a method of achieving at least one result in a healthy elderly human, the at least one result selected from the group consisting of (i) increasing levels of free reduced glutathione, (ii) increasing the ratio of free reduced glutathione to oxidized glutathione, (iii) modulating whole-blood concentrations of glycine and cysteine, (iv) minimizing or preventing lipid oxidation, (v) reducing markers of oxidative damage, and (vi) shifting fuel oxidation toward fatty acids, the method comprising: orally administering to the healthy elderly human an effective amount of a nutritional product comprising at least one glycine or functional derivative thereof, at least one N-acetylcysteine or functional derivative thereof, a buffer salt, and an ingredient selected from the group consisting of a protein, an acidic component, and a mixture thereof, wherein the nutritional product has been produced before the administering by a process comprising reconstituting a powder into a liquid comprising the ingredient, the powder comprising the at least one glycine or functional derivative thereof, the at least one N-acetylcysteine or functional derivative thereof, and the buffer salt.

In some embodiments, the methods disclosed herein include the reconstitution step. In these embodiments, the reconstitution step is preferably performed by an individual who is the recipient of the administering and/or performs the administering, and/or another individual acting under the direction of individual who is the recipient of the administering and/or performs the administering. The liquid in which the powder is reconstituted preferably contains an acidic component (e.g., juice and/or a polyphenol such as a flavonoid) and/or a protein (e.g., a milk protein such as whey and/or casein; plant protein; collagen; and blends thereof).

The present disclosure also provides a kit comprising at least one glycine or functional derivative thereof, at least one N-acetylcysteine, L-cysteine or functional derivative thereof, and buffer salt in one or more container. In some embodiments, one or more of these compounds can be isolated compounds. The kit preferably includes instructions for reconstituting the mixture in a liquid comprising at least one of an acidic component (e.g., juice and/or a polyphenol such as a flavonoid) or a protein (e.g., a milk protein such as whey and/or casein; plant protein; collagen; and blends thereof).

In an embodiment of the kit, the at least one glycine or functional derivative thereof, the at least one N-acetylcysteine, L-cysteine or functional derivative thereof, and the buffer salt can be provided together in one or more prepackaged unit dosage forms, for example in separate containers that each contain a dried powder such that each container contains one prepackaged unit dosage form.

In another embodiment, the kit can comprise a plurality of compositions for admixing together to form one or more of the powders disclosed herein. For example, the kit can contain two or more dried powders in separate containers relative to each other, the separate powders each containing a portion of the final unit dosage form.

As a non-limiting example of such an embodiment, the kit can contain one or more first containers that house the at least one glycine or functional derivative thereof and can also contain one or more second containers that house the at least one N-acetylcysteine or functional derivative thereof. The content of one of the first containers can be admixed with one of the second containers to form at least a portion of the unit dosage form of the composition. In such an embodiment, the kit can have a configuration selected from the group consisting of (i) the buffer salt is provided by the selected first container, (ii) the buffer salt is provided by the selected second container, (iii) a portion of the buffer salt is provided by the selected first container and another portion of the buffer salt is provided by the selected second container, and (iv) the buffer salt is provided by at least one of a plurality of third containers.

As used herein, a “functional derivative” of glycine is a glycine derivative that is effective in an individual in conjunction with N-acetylcysteine or a functional derivative thereof to increase intracellular GSH levels. A “functional derivative” of N-acetylcysteine is an N-acetylcysteine derivative that is effective in an individual in by itself or in conjunction with glycine (or a functional derivative thereof) to increase intracellular GSH levels.

The glycine is preferably L-glycine and/or L-glycine ethyl ester. Non-limiting examples of suitable glycine functional derivatives include D-Allylglycine; N-[Bis(methylthio)methylene]glycine methyl ester; Boc-allyl-Gly-OH (dicyclohexylammonium) salt; Boc-D-Chg-OH; Boc-Chg-OH; (R)—N-Boc-(2′-chlorophenyl)glycine; Boc-L-cyclopropylglycine; Boc-L-cyclopropylglycine; (R)—N-Boc-4-fluorophenylglycine; Boc-D-propargylglycine; Boc-(S)-3-thienylglycine; Boc-(R)-3-thienylglycine; D-a-Cyclohexylglycine; L-a-Cyclopropylglycine; N-(2-fluorophenyl)-N-(methylsulfonyl)glycine; N-(4-fluorophenyl)-N-(methylsulfonyl)glycine; Fmoc-N-(2,4-dimethoxybenzyl)-Gly-OH; N-(2-Furoyl)glycine; L-a-Neopentylglycine; D-Propargylglycine; sarcosine; Z-a-Phosphonoglycine trimethyl ester, and a mixture thereof.

The powder and/or the resultant nutritional product can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

The glycine and the N-acetylcysteine or L-cysteine or functional derivative may be formulated in a particular ratio. In some embodiments, the formulation may comprise these components in the following exemplary ratios: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, 1:150, 1:200, 1:300, 1:400, 1:500, 1:600, 1:750, 1:1000, and 1:10,000, and each of these ratios can be GLY:NAC in some embodiments and NAC:GLY in other embodiments. In particular embodiments, the formulation may comprise these components in the following weight percentages (either the same for both glycine and the N-acetylcysteine or different weight percentages for each): 1%, 2%, 3%, 4%, 5%, 6%, 7%, 8%, 9%, 10%, 12%, 15%, 20%, 25%, 30%, 40%, 45%, 50%, 55%, 60%, 65%, 70%, 75%, 80%, 85%, 90%, 95%, 97%, or 99%, for example.

The N-acetylcysteine or functional derivative thereof can be administered in an amount of about 0.1-100 milligram (mg) of N-acetylcysteine (NAC) or functional derivative thereof per kilogram (kg) of body weight of the subject. The glycine (GLY) or functional derivative thereof can be administered in an amount of about 0.1-100 milligram (mg) of glycine or functional derivative thereof per kilogram (kg) of body weight of the subject. In some embodiments, these amounts are provided at least partially by a dipeptide comprising both the N-acetylcysteine or functional derivative thereof and the glycine or functional derivative thereof.

In a particular non-limiting example, the daily doses for a 60 kg subject can be as follows:
NAC or functional derivative thereof: 6 to 6,000 mg/day
GLY or functional derivative thereof: 6 to 6,000 mg/day

The ratio of the buffering salts to the amino acids can depend on the particular application of the resultant product (e.g., whether the reconstituted liquid is a beverage or some other composition category), the serving size of the resultant product, and the protein content of the resultant product. However, one of skill in this art will be able to use the disclosures herein to determine the appropriate ratio of the buffering salts to the amino acids by considering these factors.

The powder and/or the resultant nutritional product disclosed herein is preferably administered to the subject orally. As such, non-limiting examples of the form of the composition include natural foods, processed foods, natural juices, concentrates and extracts, liposomes, and sustained-release preparations.

The powder and/or the resultant nutritional product disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms. As such, administration of the composition can be achieved in various ways, but preferably oral.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, or granules, for example, with conventional additives, such as lactose, mannitol, maltodextrin, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Example

The following non-limiting example conducted by the present inventors further support the compounds, compositions, and methods disclosed herein.

### Background

Standard protein or amino acids in powder format do not have buffer salts, except for “therapeutic/active” proteins such as enzymes or antibodies. For example, these particular compounds are not stable in pure form and thus need additional salts to stabilize. In contrast, amino acids are provided without buffer salts.

Buffer salts are also common in many ready-to-drink protein applications with the additional use of basic solutions, such as potassium hydroxide or sodium hydroxide, because ready-to-drink beverages are typically heat-treated. Nevertheless, powder products that will be reconstituted in room temperature or cold temperature liquids immediately before consumption are not provided with buffer salts.

Therefore, the present inventors investigated a solution for a reconstitution of amino acids such as glycine and/or NAC in a liquid that is acidic and/or contains protein, e.g., for at-home reconstitution of an amino acid powder.

### Experiments

The present inventors performed reconstitution of various amino acid powders at room temperature, without heat processing. The amino acids were blended with or without buffer salts and then poured into the drink.

Specifically, 600 mg N-acetyl cysteine (NAC) and 600 mg Glycine were added into 100 ml water and mixed. Then 27.5 g of an unflavoured powder containing milk protein was added and mixed. In another sample, 200 mg disodium phosphate and 300 mg potassium phosphate dibasic were blended with 600 mg NAC and 600 mg Glycine. The blend was then added into 100 ml water and mixed. After this, 27.5 gram of the unflavoured powder containing milk protein was added and mixed. Use of disodium phosphate and potassium phosphate dibasic significantly decreased protein denaturation and precipitation.

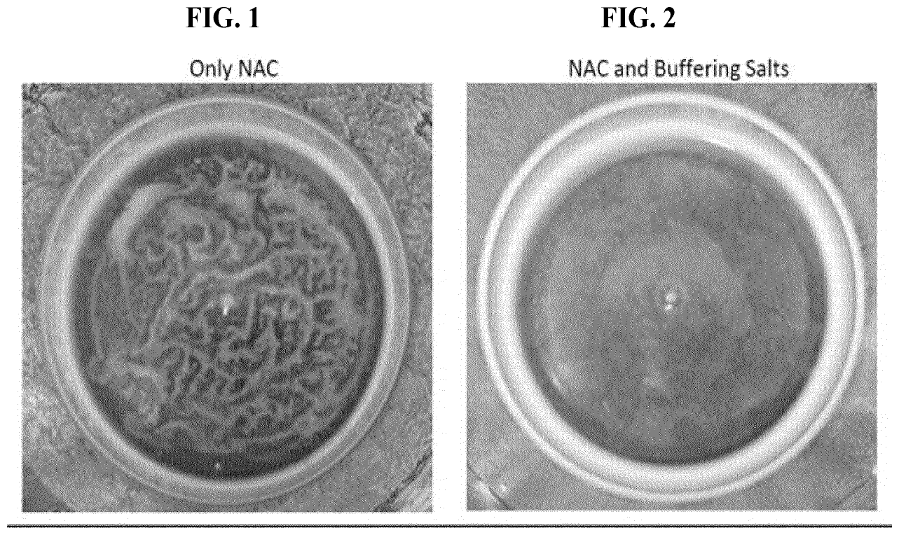

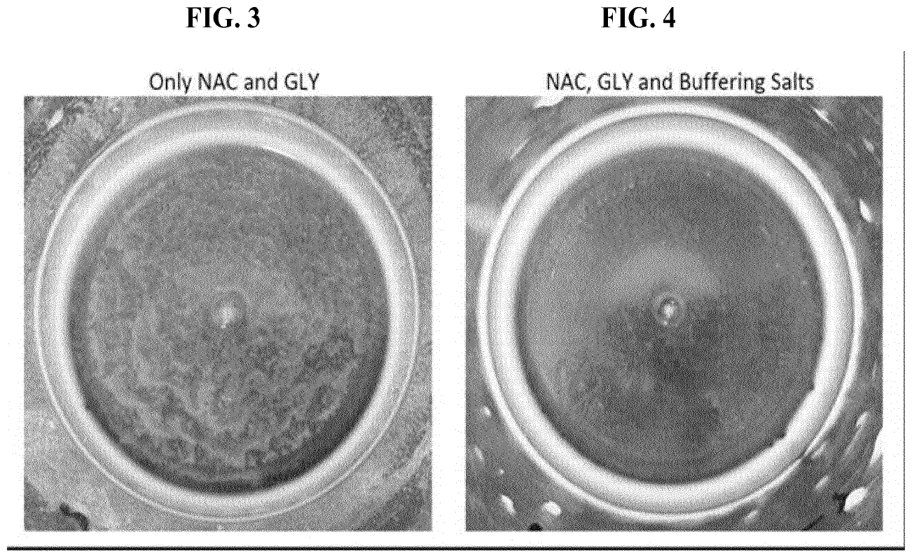

In this regard, the photographs FIGS. 1-4 show the bottom of the cup. FIG. 1 shows the result of reconstitution of powdered NAC without buffer salts, FIG. 2 shows the result of reconstitution of powdered NAC with buffer salts, FIG. 3 shows the result of reconstitution of powdered NAC and glycine without buffer salts, and FIG. 4 shows the result of reconstitution of powdered NAC and glycine with buffer salts. As shown in the figures, residues are present when the buffer salts are absent, and the presence of buffer salts avoids precipitation.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A powder comprising a phosphate salt, at least one glycine or functional derivative thereof, at least one N-acetylcysteine, L-cysteine or functional derivative thereof, Vitamin E, Vitamin C, Vitamin B2 and Zinc, the powder having at least one characteristic selected from the group consisting of (i) the amino acids in the powder are present in an amount that at least partially comprises the amino acid in a form selected from the group consisting of free form, dipeptides, tripeptides and mixtures thereof and (ii) the powder is substantially free of enzymes and antibodies.

2. The powder according to claim 1, wherein the powder comprises less than 1.0 wt. % of proteins of four or more amino acid residues.

3. The powder according to claim 1, wherein the phosphate salt comprises at least one of dibasic sodium phosphate, monobasic potassium phosphate, dipotassium phosphate, monobasic calcium phosphate, dicalcium phosphate, tricalcium phosphate, disodium phosphate or potassium phosphate dibasic.

4. The powder according to claim 3, wherein the phosphate salt comprises at least one of disodium phosphate or potassium phosphate dibasic.

5. The powder according to claim 1, wherein the powder is in a unit dosage form comprising each of the Vitamin E, the Vitamin C, the Vitamin B2 and the Zinc in an amount within the recommended daily dietary allowance.

6. The powder according to claim 5, wherein the powder in unit dosage form comprises about 600 mg N-acetylcysteine, about 600 mg glycine, and about 500 mg total phosphate salt.
